(19) United States
(12) Patent Application Publication
(10) Pub. No.: US 2023/0190149 A1
(43) Pub. Date: Jun. 22, 2023

(54) IN VIVO REDOX POTENTIAL MEASUREMENT DEVICE AND IN VIVO REDOX POTENTIAL MEASUREMENT METHOD, AND IN VIVO REDOX POTENTIAL VERIFICATION METHOD

(71) Applicant: Teruo KIYAMA, Tokyo (JP)

(72) Inventor: Teruo KIYAMA, Tokyo (JP)

(21) Appl. No.: 17/916,719

(22) PCT Filed: Apr. 14, 2021
(86) PCT No.: PCT/JP2021/015419

§ 371 Date: Oct. 3, 2022

(30) Foreign Application Priority Data
Apr. 22, 2020 (JP) 2020-076148

(51) Int. Cl. A61B 5/145

(52) U.S. Cl. CPC A61B 5/14539; A61B 5/742

(57) ABSTRACT

According to the present invention, the temperature is set to 37° C. (310 K) with respect to an equation that gives a redox potential E according to the Nernst equation, and the resulting redox potential E of hydrogen/hydrogen ion is E=−0.061×pH+0.031×pH₂ (V). At pH=7.4, E=−0.451+0.031×pH₂ (V). A hydrogen gas index pH₂ of a subject is measured, and E is calculated using the above equation. According to a potential verification method according to the present invention, the potential before and after blow-in is measured by blowing air and air containing hydrogen gas into a phosphate buffer solution, and correction is carried out as necessary to obtain a linear regression line, whereby a change in a potential associated with changes in the hydrogen gas index pH₂ can be checked and the redox potential obtained by the calculation can be recognized as being close to an actual redox potential.

### TECHNICAL FIELD

The present invention relates to an in vivo redox potential measurement device and an in vivo redox potential measurement method, and an in vivo redox potential verification method.

### BACKGROUND ART

Most of the in vivo metabolic reactions are based on oxidation-reduction reactions accompanied by electron movement. Redox is a compound word for reduction and oxidation, and redox potential is defined by rates of an oxidized type substance and a reduced type substance (a redox pair) in the equilibrium state of the half reaction of the oxidation-reduction reaction accompanied by the electron transfer. The Nernst equation is as follows.

aOx+ne− [symbol] bRed

(Ox and Red are respectively an oxidant and a reductant in an oxidation-reduction system, e− is an electron, and a, b, and n are the number of molecules or electrons. Equilibrium electrode potential E is, E=E⁰+(RT/nF)In([Ox]^{a}/[Red]^{b}). Here, E⁰ is the standard electrode potential with respect to the standard hydrogen electrode. As described in Non-Patent Document 1 below, in 1967 Williamson et al. measured the ratio of nicotinamide dinucleotide NADH/NAD⁺ in individual metabolites to measure the redox potential thereof. In addition, in 2001, Shapiro et al. directly measured the concentration of the individual glutathione 2GSH/GSSG to measure the redox potential thereof. As described in Non-Patent Document 2 below, in 2002, Jones et al. reported on the biological significance of the redox potential that the redox potential of cultured cells is lowest in the proliferative phase of the cells, intermediate in the differentiation phase (the steady state), and becomes high in the cell death of apoptosis, and cell function is regulated by controlling the redox state.

As described in Non-Patent Document 3 below, in 2019, Hatori pointed out that a correlation is observed between the oxidation-reduction state of the intracellular glutathione and various diseases, and it is more intuitive and convenient to treat the scale of the oxidation-reduction balance by the redox potential (mV) rather than the glutathione concentration ratio (%). The oxidation-reduction balance and the redox state have been studied as oxidative stress due to reactive oxygen and the like, and the intracellular localization of a glutathione oxidation sensor has been examined by a GFP fluorescence method. Furthermore, it has been reported that the redox state controls transcription and expression of genes, localization, synthesis, and degradation of intracellular substances, as well as proliferation and differentiation of cells and cell death.

The in vivo pH is strictly controlled by the regulation of carbon dioxide by respiration and metabolism in the kidney, and the normal value thereof is 7.35 to 7.45. By the way, the hydrogen in the exhaled breath is a hydrogen gas discharged from the pulmonary circulation, which has undergone the pulmonary circulation after being produced by the intestinal bacteria. The device for measuring the hydrogen gas concentration in exhaled breath belongs to Class II medical instruments, one of which is a handy type Gastrolyser (Bedfont Scientific Ltd., Kent, UK). It has been pointed out that the hydrogen gas concentration in exhaled breath fluctuates depending on diet and exercise. As described in Non-Patent Document 4 below, according to the 2017 North American Consensus, the hydrogen exhaled breath test that is carried out for the diagnosis of gastrointestinal tract disease is a test of measuring the hydrogen gas concentration in the exhaled breath for 2 hours or more, which is the transit time in the gastrointestinal tract, after fasting overnight to rule out the influence of diet and then taking a test meal and being in a rest state. After pulmonary circulation, due to gas exchange in the lung, the level of the hydrogen gas produced in the gastrointestinal tract in the mixed venous blood decreases to the hydrogen gas partial pressure in the pulmonary alveoli. This hydrogen gas partial pressure in the pulmonary alveoli is equal to the hydrogen gas partial pressure of the exhaled breath at the final stage. The hydrogen gas in the blood is carried from the pulmonary circulation to the tissues of the whole body via systemic circulation and diffuses into cells. Since the hydrogen gas is an inert gas and is not metabolized, the hydrogen gas partial pressure in the tissue is equal to the hydrogen gas partial pressure of the exhaled breath at the final stage. It almost reaches an equilibrium state (1−(½)⁴=94%) in 2 hours, which is four times the semi-saturation time, since the semi-saturation time of the inert gas (nitrogen) in the body is about 30 minutes.

### PRIOR ART DOCUMENTS

### Non-Patent Documents

Non-Patent Document 1: Williamson, D. H., Lund, P. & Krebs, H. A. The redox state of free nicotinamide-adenine dinucleotide in the cytoplasm and mitochondria of rat liver. The Biochemical journal 103, 514-527 (1967).
Non-Patent Document 2: Jones, D. P. Redox potential of GSH/GSSG couple: assay and biological significance. Methods in enzymology 348, 93-112 (2002).
Non-Patent Document 3: Yuta Hatori. Construction of gastrointestinal tract inflammation model and application of intracellular oxidation-reduction sensor. YAKUGAKU ZASSHI 139, 1523-1530 (2019).
Non-Patent Document 4: Rezaie, A., et al. Hydrogen and Methane-Based Breath Testing in Gastrointestinal Disorders: The North American Consensus. The American journal of gastroenterology 112, 775-784 (2017).

### SUMMARY OF THE INVENTION

A redox state is measured as a redox potential from a ratio or concentration of an intracellular redox pair or from the fluorescence thereof. Since the oxidation state and reduction state of the same substance cannot be distinguished, the NADH/NAD+ ratio was measured from the ratio of metabolites, and the redox potential has been measured. In addition, since glutathione forms a dimer in the oxidized type glutathione disulfide, it has been necessary to measure the concentration thereof. Although this measurement is possible in cultured cells, it has been difficult to measure the redox potential for evaluating the in vivo oxidation-reduction state since it is necessary to measure the rate, concentration, and fluorescence of metabolites in the cytoplasm. It is difficult to directly measure the in vivo redox potential such as a human in the related art, and thus it has been required to measure the redox potential, which serves as an indicator of human health.

The redox potential can be measured in a case where the in vivo redox reaction is in an equilibrium state. The equilibrium state between a hydrogen gas and hydrogen ions, which is one of the redox pairs, is as follows.

2H⁺+2e⁻ [symbol] H₂

The standard hydrogen electrode is a platinum electrode obtained by being immersed in a solution having a hydrogen ion activity of 1 Mol/L, into which a hydrogen gas has been blown at the standard pressure P⁰ (101.3 kPa). Here, the standard electrode potential E⁰=0 V. The Nernst equation is as follows.

aOx+ne− [symbol] bRed

(Ox and Red are respectively an oxidant and a reductant in an oxidation-reduction system, e− is an electron, and a, b, and n are the number of molecules or electrons. Equilibrium electrode potential E is, E=E⁰+(RT/nF)In([Ox]^{a}/[Red]^{b}) That is, the redox potential E according to the Nernst equation is E=0−(0.061/2)×(log[H₂]−2 log[H⁺]) at a temperature of 37° C. (310 K). Regarding the logarithm of the reciprocal of the hydrogen ion activity [H⁺], pH=−log[H⁺] is established. Although a hydrogen gas can be blown at the standard pressure P⁰, the hydrogen gas dissolves in an aqueous solution according to the Henry's law even in a case where the level thereof is lower than the standard pressure. As a result, the hydrogen gas activity [H₂] is, “the hydrogen gas partial pressure/the standard pressure P⁰”. Here, a hydrogen gas index pH₂=−log[H₂] is defined, where it is the logarithm of the reciprocal of the hydrogen gas activity [H₂]. The redox potential E of hydrogen/hydrogen ion is E=−0.061×pH+0.031×pH₂ (V). The redox potential depends on pH and pH₂. The rates of change in potential thereof are 0.061 V/pH and 0.031 V/pH₂.

In the present invention, the hydrogen gas partial pressure in the exhaled breath at the final stage is measured, and the hydrogen gas index pH₂, which is the logarithm of the reciprocal of “the hydrogen gas partial pressure/the standard pressure”, is displayed by the device. Regarding the redox potential, the redox potential E is measured according to the calculation equation E=−0.451+0.031×pH₂ (V) by using the body temperature of 37° C. (310 K), the normal pH value of 7.4 of the living body, and the hydrogen gas index pH₂.

The in vivo redox potential measurement device according to the present invention is a device that measures the in vivo redox potential by the hydrogen gas analysis in the exhaled breath and displays the result as a voltage. The in vivo redox potential measurement method according to the present invention measures the in vivo redox potential by the hydrogen gas analysis in the exhaled breath and displays the result as a voltage. The in vivo redox potential (V) is measured using the in vivo ratio of the hydrogen to the hydrogen ion. The redox potential is defined by the ratio between an oxidized type substance and a reduced type substance (a redox pair) in the equilibrium state of the half reaction of the oxidation-reduction reaction accompanied by the electron transfer. Here, the potential of the standard hydrogen electrode is determined as the reference point 0 V by using the Nernst equation for the equilibrium electrode potential.

For the hydrogen gas analysis, a device for measuring the hydrogen gas partial pressure in the pulmonary alveoli with the exhaled breath at the final stage under atmospheric air pressure is included. The hydrogen gas activity [H₂] is proportional to the hydrogen gas partial pressure (Pa)/101.3 kPa since a state in which a hydrogen gas at the standard pressure P⁰ is blown at all times at the standard hydrogen electrode is set to 1. The result of the hydrogen gas analysis is expressed by defining the hydrogen gas index pH₂ as the logarithm of the reciprocal of [H₂] (pH₂=−log[H₂]).

Further, the in vivo redox potential verification method according to the present invention verifies that an estimated value is close to the actual in vivo redox potential that would be obtained in a case of being actually measured since the in vivo redox potential measured by the in vivo redox potential measurement device and the in vivo redox potential measurement method according to the present invention is not an actual measured value but is based on a predetermined calculation equation and thus it is merely the estimated value in a sense.

According to the present invention, there is provided an in vivo redox potential measurement device including, in a case where a hydrogen gas index pH₂ is defined as a logarithm of a reciprocal of hydrogen gas partial pressure (Pa)/101.3 kPa (pH₂=−log[hydrogen gas partial pressure/101.3 kPa]); a means that measures a hydrogen gas index pH₂ of a subject of a person to be subjected to measurement,

a storage means that stores a predetermined calculation equation;

a calculation means that calculates a redox potential according to the predetermined calculation equation stored in the storage means, by using the hydrogen gas index pH₂ measured by the means that measures the hydrogen gas index; and

a display means that displays the redox potential calculated by the calculation means.

In the in vivo redox potential measurement device according to the present invention, one preferred embodiment is using E=−0.451+0.031×pH₂ (V) as the calculation equation in a case where E is denoted as the redox potential and pH₂ is denoted as the hydrogen gas index.

In the in vivo redox potential measurement device according to the present invention, one preferred embodiment is using E=−0.061×pH+0.031×pH₂ (V) as the calculation equation in a case where E is denoted as the redox potential, pH is denoted as the hydrogen ion index of the subject, and pH₂ is denoted as the hydrogen gas index.

In the in vivo redox potential measurement device according to the present invention, one preferred embodiment is using a value of 7.3 to 7.5 as the pH.

In the in vivo redox potential measurement device according to the present invention, one preferred embodiment is using a value of 7.4 as the pH.

In the in vivo redox potential measurement device according to the present invention, one preferred embodiment is configuring the display means to display the hydrogen gas index pH₂ measured by the means that measures the hydrogen gas index.

According to the present invention, there is provided an in vivo redox potential measurement method including, in a case where a hydrogen gas index pH₂ is defined as a logarithm of a reciprocal of hydrogen gas partial pressure (Pa)/101.3 kPa (pH₂=−log[hydrogen gas partial pressure/101.3 kPa]); a step of measuring a hydrogen gas index pH₂ of a subject of a person to be subjected to measurement;

s step of reading out a predetermined calculation equation stored in a predetermined storage means;

a calculation step of calculating a redox potential according to the predetermined calculation equation read out from the storage means, by using the hydrogen gas index pH₂ measured by the means that measures the hydrogen gas index; and

a display step of displaying the redox potential calculated in the calculation step.

In the in vivo redox potential measurement method according to the present invention, one preferred embodiment is using E=−0.451+0.031×pH₂ (V) as the calculation equation in a case where E is denoted as the redox potential and pH₂ is denoted as the hydrogen gas index.

In the in vivo redox potential measurement method according to the present invention, one preferred embodiment is using E=−0.061×pH+0.031×pH₂ (V) as the calculation equation in a case where E is denoted as the redox potential, pH is denoted as the hydrogen ion index of the subject, and pH₂ is denoted as the hydrogen gas index.

In the in vivo redox potential measurement method according to the present invention, one preferred embodiment is using a value of 7.3 to 7.5 as the pH.

In the present invention, one preferred embodiment is using a value of 7.4 as the pH.

In the in vivo redox potential measurement method according to the present invention, one preferred embodiment is configuring the display step to display the hydrogen gas index pH₂ measured in the step of measuring the hydrogen gas index.

According to the present invention, there is provided an in vivo redox potential verification method which is for verifying that an in vivo redox potential determined by a measured hydrogen gas index pH₂ and a predetermined calculation equation is close to an actual in vivo redox potential, the in vivo redox potential verification method comprising, in a case where a hydrogen gas index pH₂ is defined as a logarithm of a reciprocal of hydrogen gas partial pressure (Pa)/101.3 kPa (pH₂=−log[hydrogen gas partial pressure/101.3 kPa]);

a first step of putting a phosphate buffer solution in a container constituting a bubbling device;

a second step of measuring a pH and an oxidation-reduction potential ORP of the phosphate buffer solution in the container;

a third step of carrying out air sending of medical air to the bubbling device at a first predetermined flow rate for a first predetermined time;

a fourth step of measuring again the pH and the oxidation-reduction potential ORP of the phosphate buffer solution in the container, after completion of the air sending for the predetermined time;

a fifth step of correcting the oxidation-reduction potential ORP measured after carrying out the air sending of the medical air, with the oxidation-reduction potential ORP measured before carrying out the air sending of the medical air;

a sixth step of discharging the phosphate buffer solution in the container and putting a new phosphate buffer solution in the container;

a seventh step of measuring a pH and an oxidation-reduction potential ORP of the new phosphate buffer solution in the container;

an eighth step of carrying out air sending of a standard gas including hydrogen having a predetermined concentration, to the bubbling device at a second predetermined flow rate for a second predetermined time;

a ninth step of measuring again the pH and the oxidation-reduction potential ORP of the new phosphate buffer solution in the container, after completion of the air sending for the second predetermined time;

a tenth step of correcting the oxidation-reduction potential ORP measured after carrying out the air sending of the standard gas including the hydrogen having the predetermined concentration, with the oxidation-reduction potential ORP measured before carrying out the air sending of the standard gas including the hydrogen having the predetermined concentration; and

an eleventh step of using the two corrected oxidation-reduction potentials ORP, obtained in the two steps of carrying out the correction, to grasp a change aspect of the oxidation-reduction potential ORP with respect to a change of pH₂ by setting the pH₂ to 6.2 in a case of the medical air and setting the pH₂ to 4 in a case of the standard gas including the hydrogen having the predetermined concentration.

In the in vivo redox potential verification method according to the present invention, one preferred embodiment repeating the first step to the fifth step a plurality of times and using an average value of measurement results from the plurality of times of repetition, and similarly repeating the sixth step to the tenth step a plurality of times and using an average value of measurement results from the plurality of times of repetition.

A living body receives changes in temperature, nutrients, oxygen, and the like as signals from the outside world as an environment. In addition, the living body receives a change in the relatively hypoxic state or the lack of glucose as a substrate, as an endogenous signal, due to changes in metabolic demands, and it controls cell functions to maintain constant homeostasis in the living body. One of these signal transductions is oxidative stress, including reactive oxygen species. Reduction systems such as glutathione and thioredoxin are present in a living body, which maintain homeostasis by receiving energy supply from NADH and NADPH. Such an oxidation-reduction reaction state is called a redox state in a living body and is measured as a redox potential. The redox potential is defined by each redox pair, and the intracellular concentration of glutathione, which is one of the redox pairs, is 3 to 10 mM, and glutathione has a buffering function due to the large amount thereof. On the other hand, the concentration of NADH is 97 to 168 μM, and NADH is always reused between the TCA cycle and the electron transport chain. That is, in a case where these redox pairs are used as an indicator, the change in the redox potential is not sensitive, and thus there is no change in the potential except for a proliferation state, differentiation, and apoptosis of cultured cells, in which the metabolic state changes significantly.

In the present invention, the in vivo oxidation-reduction state is evaluated by using the redox potential of the hydrogen/hydrogen ion, which is one of the indicators of the oxidation-reduction state. The in vivo hydrogen ion concentration is 40 nM at pH=7. In a case where the hydrogen concentration in the exhaled breath test is 13 ppm (6 to 20 ppm in an early morning fasting state), the in vivo hydrogen concentration is 10 nM from the solubility of the hydrogen gas (1,282 L×atm/mol). That is, it is one millionth of glutathione and one thousandth or less of NADH. The hydrogen ion concentration in cations of the blood in acid-base balance is as low as 40 nM, which is one millionth of the sodium ion concentration of 130 mEq/L, and it is sensitive to changes. It is suggested that the change in the redox potential in a case of using the hydrogen/hydrogen ion as an indicator of the oxidation-reduction balance is sensitive by 1,000 to 1 million times as compared with a case of using glutathione or NADH as an indicator.

The present invention makes it possible to more sensitively capture short-term changes in metabolic state. So far, only long-term changes in proliferation and differentiation of cells and cell death could be captured. A sensitive indicator that is capable of being repeatedly measured can capture changes in the environment as the outside world and changes in the endogenous metabolic demands. For example, no change was shown in the hydrogen exhaled breath test by fasting and diet, or rest and exercise. In addition, due to the fact that the hydrogen partial pressure in the body reaches equilibrium in about 2 hours, it is possible to measure the influence of a disease or a drug for treatment by using the in vivo redox potential as an indicator. As a result, in a case where the redox potential can be measured, it is conceived that it is applicable not only to the evaluation of the environment and metabolic state but also to the evaluation of diseases and the development of treatment methods.

### BRIEF DESCRIPTION OF THE DRAWINGS

FIG. 1 is a block diagram illustrating a preferred embodiment of an in vivo redox potential measurement device according to the present invention.

FIG. 2 is a block diagram illustrating a preferred embodiment of a system that is used in an in vivo redox potential verification method according to the present invention.

FIG. 3 is a graph for describing the in vivo redox potential verification method according to the present invention.

### MODES FOR CARRYING OUT THE INVENTION

Hereinafter, preferred embodiments according to the present invention will be described with reference to the drawings.

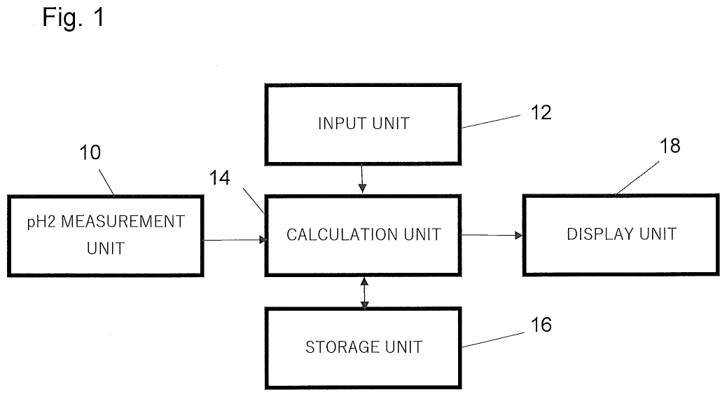

FIG. 1 is a block diagram illustrating a preferred embodiment of an in vivo redox potential measurement device according to the present invention. A preferred embodiment of an in vivo redox potential measurement method according to the present invention is also described according to FIG. 1.

In the present invention, the in vivo redox potential of a person as a person to be measured is measured. However, in the present invention, it is intended to use an exhaled breath of the person to be measured since it is difficult to directly measure the redox potential, for example, in the blood, internal organs, muscles, and the like of the living body due to electrical resistance. A pH₂ measurement unit 10 measures the hydrogen gas index pH₂ in the exhaled breath in a case where a test target blows an exhaled breath. As the pH₂ measurement unit 10, it is possible to use, for example, a semiconductor-type hydrogen concentration meter such as SG8541 manufactured by RIKEN KEIKI Co., Ltd., where it detects a change in electric resistance value, which occurs in a case where a metal oxide semiconductor to be heated comes into contact with the hydrogen gas, as a hydrogen gas concentration. The output signal of the pH₂ measurement unit 10 is an output signal that indicates the measured hydrogen gas index pH₂, and it is sent out to a calculation and control unit 14. A predetermined calculation equation is stored in advance in a storage unit 16, and it stores a calculation result of the calculation and control unit 14 as necessary. An input unit 12 is used to give an instruction of calculation start to the calculation and control unit 14 and to set a value of the hydrogen ion index pH described later in the calculation and control unit 14.

The in vivo redox potential calculated by the calculation and control unit 14 is sent out to a display unit 18 and displayed as a numerical value. It is noted that the input unit 12, the calculation and control unit 14, the storage unit 16, and the display unit 18 can also consist of a keyboard and mouse of a personal computer, a central processing unit (CPU), a memory (RAM, ROM), and a display, respectively. In this case, in order to supply the output signal of the pH₂ measurement unit 10 to a USB input unit of the personal computer in an appropriate format, an interface (not illustrated in the drawing) is used as necessary. In addition to displaying the in vivo redox potential, the display unit 18 can also display the measured hydrogen gas index pH₂. It is noted that in addition to the above calculation, the calculation and control unit 14 can control the storage unit 16 and the display unit 18 according to an instruction from an operator of a device to which the instruction has been input by the input unit 12.

The storage unit 16 stores in advance a calculation equation for calculating the in vivo redox potential E. The basis of the calculation equation is E=−0.061×pH+0.031×pH₂ (V). Here, since the pH is 7.35 to 7.45 as described above, an appropriate value within this range can be input into the calculation unit by the input unit 12. On the other hand, since the pH change range is as small as 7.35 to 7.45, this can be fixed to, for example, 7.4. As a result, 7.4 can be input as the pH by the input unit 12, or 7.4 can be stored in the storage unit 16 in advance and then used. However, the above-described basic calculation equation can be simplified to E=−0.451+0.031×pH₂ (V) by incorporating 7.4 in advance as the pH. As a result, in a case where the pH is fixed to 7.4, the simplified calculation equation E=−0.451+0.031×pH₂ (V) can be stored in the storage unit 16 instead of the basic calculation equation. It is noted that both the basic calculation equation and the simplified calculation equation can be stored in the storage unit 16, and as necessary, one of them can be read out by the instruction from the input unit 12 and given to the calculation unit 14.

Next, some actual measurement examples of the redox potential measured using the above-described simplified calculation equation are shown.

Measurement conditions, measurement target person, and like

Measurement date and time: Sep. 26, 2018, 8 o'clock to 9 o'clock in the morning

A case 1; A 23-year-old woman with breakfast, hydrogen gas partial pressure: 0.1 Pa, hydrogen gas index pH₂=6.0, redox potential E=−0.265 V.

A case 2: A 44-year-old man without breakfast, hydrogen gas partial pressure: 1.9 Pa, hydrogen gas index pH₂=4.72, redox potential E=−0.305 V.

A case 3: A 34-year-old woman with breakfast, hydrogen gas partial pressure: 4.6 Pa, hydrogen gas index pH₂=4.34, redox potential E=−0.317 V.

The table below shows the measurement results of a total of 10 cases including the above cases 1 to 3.

TABLE 1

| | | | Hydrogen | | Redox |
|---|---|---|---|---|---|
| | | | partial | | potential |
| | | | pressure | | E |
| Number | Age | Sex | (Pa) | pH₂ | (V) |
| 1 | 23 | Female | 0.1 | 6.000 | −0.265 |
| 2 | 44 | Male | 1.9 | 4.721 | −0.305 |
| 3 | 34 | Female | 4.6 | 4.337 | −0.317 |
| 4 | 35 | Male | 3 | 4.523 | −0.311 |
| 5 | 23 | Male | 0.8 | 5.097 | −0.293 |
| 6 | 25 | Female | 0.5 | 5.301 | −0.287 |
| 7 | 24 | Male | 0.4 | 5.398 | −0.284 |
| 8 | 24 | Female | 2.6 | 4.585 | −0.309 |
| 9 | 22 | Female | 1.5 | 4.824 | −0.301 |
| 10 | 24 | Male | 1.4 | 4.854 | −0.301 |

Next, a preferred embodiment of the system used in the in vivo redox potential verification method according to the present invention will be described.

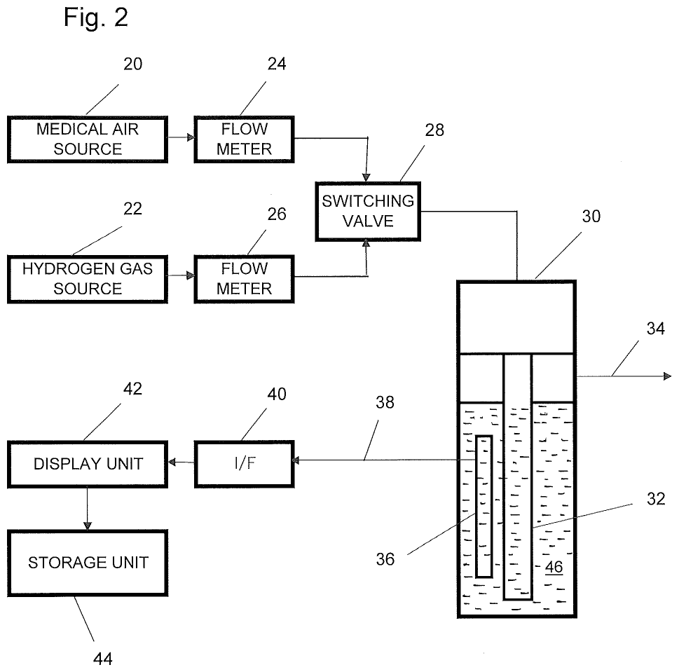

FIG. 2 is a block diagram illustrating a preferred embodiment of a system that is used in an in vivo redox potential verification method according to the present invention. This system includes a medical air source 20, a hydrogen gas source 22, two flow meters 24 and 26, a switching valve 28, a bubbling device 30, a pH and oxidation-reduction potential measurement device 36 provided in a container 31 constituting the bubbling device 30, an interface 40, a display unit 42, and a storage unit 44. As the bubbling device 30, a bubbling device that is generally used as an air humidifier can be used. It guides a gas supplied from the outside through a conduit tube 32 into the container 31, carries out the bubbling of the introduced gas (making bubbles) in the liquid in the container 31, and discharges it from the upper space of the container 31 to the outside through the discharge pipe 34.

The medical air source 20 sends out a medical air decompressed to 200 kPa. The hydrogen gas source 22 sends out air (the standard gas) containing 10 Pa of hydrogen. Any one of the decompressed medical air sent out from the medical air source 20 and the air containing hydrogen sent out from the hydrogen gas source 22 is selected by the switching valve 28 through each of the flow meters 24 and 26 and supplied to the bubbling device 30. The bubbling device 30 is a bubbling device that is generally used as an air humidifier, and a phosphate buffer solution (10 mM, pH 7.1) 46 can be put into the container 31 constituting the bubbling device 30. As such a phosphate buffer solution it is possible to use, for example, 166-23555 PBS (−) manufactured by FUJIFILM Wako Pure Chemical Corporation. A preferred embodiment of an in vivo redox potential verification method according to the present invention will be described by using FIG. 2.

A phosphate buffer solution (10 mM, pH 7.1) 46 is put in the container 31 constituting the bubbling device 30. The pH and oxidation-reduction potential ORP of the phosphate buffer solution in the container 31 are measured with the pH and oxidation-reduction potential measurement device 36. As the pH and oxidation-reduction potential measurement device 36, it is possible to respectively use, for example, pH 6600 and ORP-66005, manufactured by CUSTOM corporation. The output signal of the pH and oxidation-reduction potential measurement device 36 is given to the display unit 42 and the storage unit 44 through a signal transmission path 38 and the interface 40. In a case where the pH and reduction potential are measured and stored in the storage unit 44, the switching valve 28 is operated to carry out the air sending of the medical air to the bubbling device 30 at a first predetermined flow rate for a first predetermined time. Here, the first predetermined flow rate is set to 0.5 L/min, and the first predetermined time is set to 1 hour. As the medical air, it is possible to use, for example, medical air manufactured by AIR WATER Inc. (including 0.6 ppm of hydrogen as in the atmospheric air).

After the completion of the air sending for the first predetermined time, the pH and oxidation-reduction potential ORP of the phosphate buffer solution in the container 31 are measured again by the pH and oxidation-reduction potential measurement device 36, and the measured potential is stored in the storage unit 44. Next, the oxidation-reduction potential ORP measured after carrying out the air sending of the medical air is corrected with the oxidation-reduction potential ORP measured before carrying out the air sending of the medical air. For example, in a case where the oxidation-reduction potential ORP measured before carrying out the air sending of the medical air is 230 mV, this potential is set to 0 mV as a reference. That is, in a case where the oxidation-reduction potential ORP measured after carrying out the air sending of the medical air is 243 mV, 230 mV is subtracted from this value to correct 243 mV to 13 mV.

The phosphate buffer solution in the container 31 is discharged, and a new phosphate buffer solution is put in the container 31. The pH and oxidation-reduction potential ORP of the new phosphate buffer solution in the container 31 are measured in the same manner as described above. After the measurement and the storage of the measured values are completed, the air sending of a standard gas including hydrogen having a predetermined concentration to the bubbling device 30 is carried out at a second predetermined flow rate for a second predetermined time. Here, the second predetermined flow rate is set to 0.5 L/min, and the second predetermined time is set to 1 hour. In addition, the predetermined concentration is, for example, 100 ppm, and it is possible to use, for example, a standard gas manufactured by AIR WATER Inc.

After the completion of the air sending for the second predetermined time, the pH and oxidation-reduction potential ORP of the new phosphate buffer solution in the container 31 are measured again by the pH and oxidation-reduction potential measurement device 36 and stored. The oxidation-reduction potential ORP measured after carrying out the air sending of the standard gas including the hydrogen having the predetermined concentration is corrected with the oxidation-reduction potential ORP measured before carrying out the air sending of the standard gas including the hydrogen having the predetermined concentration. Here, in a case where the measured potential is 174 mV, 230 mV is subtracted therefrom as described above, and the corrected potential is set to −56 mV.

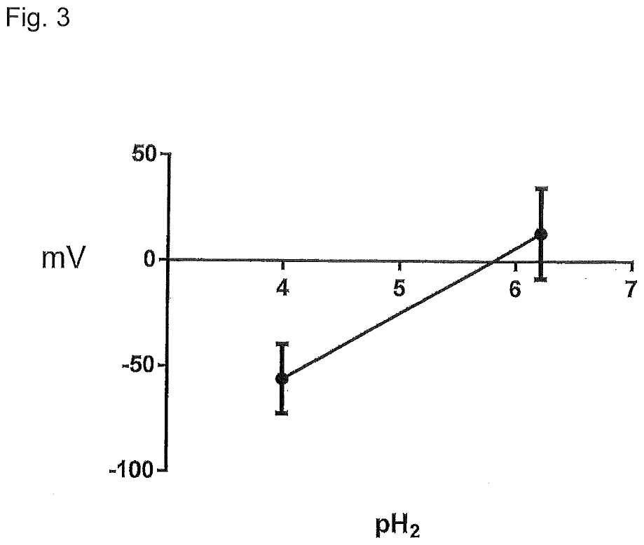

Using the two corrected oxidation-reduction potentials ORP obtained as described above, a change aspect of the oxidation-reduction potential ORP with respect to a change of pH₂ is grasped by setting the pH₂ to 6.2 in a case of the medical air and setting the pH₂ to 4 in a case of the standard gas including the hydrogen having the predetermined concentration. That is, as shown in the graph in FIG. 3, the oxidation-reduction potential ORP is −56 mV in a case where pH₂ is 4, and the oxidation-reduction potential ORP is 13 mV in a case where pH₂ is 6.2. As a result, a line segment connecting these points is obtained to create the graph in FIG. 3. The change in pH is 0.007±0.021 in a case where the air has been subjected to bubbling, and it is 0.060±0.008 in a case of the hydrogen standard gas (10 Pa). In addition, the change in oxidation-reduction potential is 13.3±17.6 mV after the air has been subjected to bubbling, and it is −56.0±13.5 mV in a case of the standard hydrogen gas.

Although the creation of the graph in FIG. 3 by the above measurement was described in the case of one measurement, it is preferable to measure the above potential a plurality of times (for example, three times or more) and take an average value of the measurement results in order to reduce the measurement error. In the graph in FIG. 3, the line segments extending above and below both ends of the line segment of the graph showing the potential at a pH₂ of 4 and the potential at a pH₂ of 6.2 each indicate the standard deviation. The linear regression line is significant (y=−180.8+32.2×pH₂, r²=0.83, p=0.011). The change after bubbling is less than 0.1 at pH=7.1 due to the phosphate buffer solution. However, the oxidation-reduction potential ORP is changed by 32 mV/pH₂ due to the partial pressure of the hydrogen in the air subjected to bubbling, which matched with the Nernst equation. As a result, the oxidation-reduction potential is proportional to the pH₂ due to the hydrogen gas partial pressure in the bubbling air in a case where the pH is constant.

In a case of examining this point, r² is a coefficient of determination in the above equation that gives the y. The value obtained by regression is an indicator for evaluating how well matching is actually obtained. The coefficient of determination r² usually takes a value in a range of 0 to 1, where the larger the value is, the more appropriately the data can be expressed. Next, in a case of considering the probability that the regression coefficient (the slope of the line) is 0 (irrelevant), the probability that the regression coefficient is zero, which is expressed by p, is less than 0.05 which is the level of significance in the present embodiment. As a result, it is shown that the change in potential is proportional to the hydrogen gas index pH₂. The coefficient of determination r² indicates how well the regression line matches the data, and p indicates the probability that the regression coefficient is 0, where it is less than 5% of the level of significance, from which it is determined to be statistically significant.

### INDUSTRIAL APPLICABILITY

According to the in vivo redox potential measurement device and the in vivo redox potential measurement method according to the present invention, the hydrogen gas index pH₂ is measured by using a human exhaled breath, which makes it possible to guess the in vivo redox potential that is difficult to be directly measured. As a result, it is possible to easily grasp the health state of outpatients, inpatients, other persons that undergo medical examinations, and the like. In addition, it is applicable not only to the evaluation of the environment and metabolic state but also to the evaluation of diseases and the development of treatment methods, and thus it is useful for the diagnosis and treatment industry, which carries out medical examinations and treatments of various diseases. Further, according to the in vivo redox potential verification method according to the present invention, it can be easily verified that the redox potential measured by the in vivo redox potential measurement device and the in vivo redox potential measurement method according to the present invention by using a standard gas, a phosphate buffer solution, and the like is close to the actual potential, and thus it is also similarly useful in the diagnosis and treatment industry.

1. An in vivo redox potential measurement device comprising, in a case where a hydrogen gas index pH₂ is defined as a logarithm of a reciprocal of hydrogen gas partial pressure (Pa)/101.3 kPa (pH₂=−log[hydrogen gas partial pressure/101.3 kPa]):
a means that measures a hydrogen gas index pH₂ of a subject of a person to be subjected to measurement;
a storage means that stores a predetermined calculation equation;
a calculation means that calculates a redox potential by the predetermined calculation equation stored in the storage means, by using the hydrogen gas index pH₂ measured by the means that measures the hydrogen gas index; and
a display means that displays the redox potential calculated by the calculation means.

2. The in vivo redox potential measurement device according to claim 1,
wherein E=−0.451+0.031×pH₂ (V) is used as the calculation equation in a case where E is denoted as the redox potential and pH₂ is denoted as the hydrogen gas index.

3. The in vivo redox potential measurement device according to claim 1,
wherein E=−0.061×pH+0.031×pH₂ (V) is used as the calculation equation in a case where E is denoted as the redox potential, pH is denoted as the hydrogen ion index of the subject, and pH₂ is denoted as the hydrogen gas index.

4. The in vivo redox potential measurement device according to claim 3,
wherein a value of 7.3 to 7.5 is used as the pH.

5. The in vivo redox potential measurement device according to claim 4,
wherein a value of 7.4 is used as the pH.

6. The in vivo redox potential measurement device according to claim 1,
wherein the display means is configured to display the hydrogen gas index pH₂ measured by the means that measures the hydrogen gas index.

7. An in vivo redox potential measurement method comprising, in a case where a hydrogen gas index pH₂ is defined as a logarithm of a reciprocal of hydrogen gas partial pressure (Pa)/101.3 kPa (pH₂=−log[hydrogen gas partial pressure/101.3 kPa]):
a step of measuring a hydrogen gas index pH₂ of a subject of a person to be subjected to measurement;
s step of reading out a predetermined calculation equation stored in a predetermined storage means;
a calculation step of calculating a redox potential by the predetermined calculation equation read out from the storage means, by using the hydrogen gas index pH₂ measured by the means that measures the hydrogen gas index; and
a display step of displaying the redox potential calculated in the calculation step.

8. The in vivo redox potential measurement method according to claim 7,
wherein E=−0.451+0.031×pH₂ (V) is used as the calculation equation in a case where E is denoted as the redox potential and pH₂ is denoted as the hydrogen gas index.

9. The in vivo redox potential measurement method according to claim 7,
wherein E=−0.061×pH+0.031×pH₂ (V) is used as the calculation equation in a case where E is denoted as the redox potential, pH is denoted as the hydrogen ion index of the subject, and pH₂ is denoted as the hydrogen gas index.

10. The in vivo redox potential measurement method according to claim 9,
wherein a value of 7.3 to 7.5 is used as the pH.

11. The in vivo redox potential measurement method according to claim 10,
wherein a value of 7.4 is used as the pH.

12. The in vivo redox potential measurement method according to claim 7,
wherein the display step is configured to display the hydrogen gas index pH₂ measured in the step of measuring the hydrogen gas index.

13. An in vivo redox potential verification method which is for verifying that an in vivo redox potential determined by a measured hydrogen gas index pH₂ and a predetermined calculation equation is close to an actual in vivo redox potential, the in vivo redox potential verification method comprising, in a case where a hydrogen gas index pH₂ is defined as a logarithm of a reciprocal of hydrogen gas partial pressure (Pa)/101.3 kPa (pH₂=−log[hydrogen gas partial pressure/101.3 kPa]):
a first step of putting a phosphate buffer solution in a container constituting a bubbling device;
a second step of measuring a pH and an oxidation-reduction potential ORP of the phosphate buffer solution in the container;
a third step of carrying out air sending of medical air to the bubbling device at a first predetermined flow rate for a first predetermined time;
a fourth step of measuring again the pH and the oxidation-reduction potential ORP of the phosphate buffer solution in the container, after completion of the air sending for the predetermined time;
a fifth step of correcting the oxidation-reduction potential ORP measured after carrying out the air sending of the medical air, with the oxidation-reduction potential ORP measured before carrying out the air sending of the medical air;
a sixth step of discharging the phosphate buffer solution in the container and putting a new phosphate buffer solution in the container;
a seventh step of measuring a pH and an oxidation-reduction potential ORP of the new phosphate buffer solution in the container;
an eighth step of carrying out air sending of a standard gas including hydrogen having a predetermined concentration, to the bubbling device at a second predetermined flow rate for a second predetermined time;
a ninth step of measuring again the pH and the oxidation-reduction potential ORP of the new phosphate buffer solution in the container, after completion of the air sending for the second predetermined time;
a tenth step of correcting the oxidation-reduction potential ORP measured after carrying out the air sending of the standard gas including the hydrogen having the predetermined concentration, with the oxidation-reduction potential ORP measured before carrying out the air sending of the standard gas including the hydrogen having the predetermined concentration; and
an eleventh step of using the two corrected oxidation-reduction potentials ORP, obtained in the two steps of carrying out the correction, to grasp a change aspect of the oxidation-reduction potential ORP with respect to a change of pH₂ by setting the pH₂ to 6.2 in a case of the medical air and setting the pH₂ to 4 in a case of the standard gas including the hydrogen having the predetermined concentration.

14. The in vivo redox potential verification method according to claim 13,
wherein the first step to the fifth step are repeated a plurality of times and an average value of measurement results from the plurality of times of repetition is used, and
the sixth step to the tenth step are similarly repeated a plurality of times and an average value of measurement results from the plurality of times of repetition is used.
